(19) United States
(12) Patent Application Publication
(10) Pub. No.: US 2023/0285204 A1
(43) Pub. Date: Sep. 14, 2023

(54) FLOCKED FIBER SURFACED MEDICAL SWAB DEVICES AND MANUFACTURING METHOD

(71) Applicant: University of Massachusetts, Boston, MA (US)

(72) Inventors: Yong K. Kim, North Dartmouth, MA (US); Armand F. Lewis, Mattapoisett, MA (US)

(21) Appl. No.: 18/016,798

(22) PCT Filed: Jul. 22, 2021
(86) PCT No.: PCT/US21/42797

§ 371 Date: Jan. 18, 2023

Related U.S. Application Data
(60) Provisional application No. 63/057,331, filed on Jul. 28, 2020.

(51) Int. Cl. A61F 13/38; A61B 10/00

(52) U.S. Cl. CPC A61F 13/385; A61B 10/0051; A45D 2200/1018

(57) ABSTRACT

A bodily fluid absorbing, fiber flocked modular medical swab includes a prefabricated pre-flocked tip having a pre-flocked braided yarn hollow tube; a pre-flocked hollow plastic tube; a pre-flocked fabric; a pre-flocked yarn band or a pre-flocked narrow fabric band/tape/ribbon base. A swab rod is attached to the pre-flocked tip.

### RELATED APPLICATIONS

This application is a national stage filing of PCT application No.: PCT/US2021/042797 filed Jul. 22, 2021, entitled FLOCKED FIBER SURFACED MEDICAL SWAB AND MANUFACTURING METHOD, which itself claims priority to U.S. Provisional Patent Application No. 63/057,331 filed Jul. 28, 2020, entitled FLOCKED FIBER SURFACED MEDICAL SWAB AND MANUFACTURING METHOD, the entire teachings of all of which are incorporated herein by reference.

### FIELD OF THE INVENTION

The invention relates to swabs and methods of manufacture.

### BACKGROUND

In this era of the COVID-19 pandemic there is a dire need for effective medical swabs used for collecting body fluid test samples from human nose, mouth and throat fluid collection for subsequent virus testing. Currently flocked medical swabs are manufactured by traditional flock processing ranges, which are not adaptable for moderate quantity fabrication of in-house for point-of-testing facilities under supply chain problems such as the U.S. encountering a shortage of COVID-19 test kits in February to May 2020. Moreover, flocked medical swabs manufacture by traditional flocking methods requires the knowledge of flock materials, flock processing, flocking technology and a significant capital investment for equipment and operational facilities.

There are many forms, sizes and designs of medical as well as cosmetic swab devices on the market. All of these designs involve the geometry of having a quantity of absorbent material (in the form of absorbent material ball, lump, “egg shaped” ball, ellipsoidal shaped, cylindrical sliver) secured to the end of a thin, semi-stiff “stick” or shaft (such as a wooden, plastic or waxed wrapped paper tightly rolled rod). In practice, the absorbent materials at the end of these “swab-sticks” are any of (a) fibrous absorbent cotton, (b) fibrous polypropylene, (c) flocked fiber (nylon/polyester), (d) foam and (e) serrated, “spindly” plastic shaped tips, and the like. Indeed, flocked fiber fibrous tipped “sticks”, rods or shafts have been used in medical swab applications for many years. As used herein, the term swab rod refers to the various sticks, shafts, handles described above. The terms modular pre-flocked tip, swab tip, swab ends, tips or bulbs are used to refer to various shapes and sizes to fit for particular medical swab use applications. These flocked fiber swabs are manufactured or factory fabricated employing traditional fiber flocking techniques in that each individual flocked swab device is fabricated by the electrostatic flocking of the fibers directly onto (uncured) adhesive coated tip of swab rods on a tray on a conveyor belt is passing under the flocking station. A batch of these adhesively end-coated “sticks” or swab rods trays are then collectively flocked with a suitable flock fiber (nylon or polyester fiber). These flocked fibers are then secured in place by curing the flock adhesive usually by heat oven or most often by a UV or LED radiation curing processing. After this curing process, the Flocked Swabs are vacuum cleaned for removing any not-adhered, loose flock fibers, and then given a disinfecting treatment of heat, ethylene oxide, hydrogen peroxide exposure or UV radiation, and finally rendered antiseptically packaged and ready for medical test use. These conventional swabs are expensive, require a factory for complete fabrication and generally do not have flexible tips. People receiving COVID-19 tests have reported that the nasal swap is sometimes quite painful.

### SUMMARY

Flocked fiber surfaced tipped medical swab device designs disclosed herein have features for easy manufacture and assembly. The simplicity of fabricating these devices for test kit fabrication/manufacture renders them readily adaptable to in-house on-demand fabrication of medical swabs, as needed, for medical health care facilities or testing labs requiring swabs.

Flocked swab tips amplify the biological sample collection surface area. For example, modular pre-flocked tips as described herein in various embodiments include fabric with nylon flock (2 denier, 1 mm long) and flock density of 200 fibers per square mm. In one embodiment, the collection surface area of a pre-flocked swab tip (typically 5 mm×15 mm) has 444 mm² with collection volume of 35 microliter. These modular pre-flocked tips have a sample collection efficiency advantage compared to non-flocked swabs.

In various embodiments, flock layer tipped medical swab configurations include a prefabricated, pre-formed swab tip section of a) flocked low cross-section hollow braided fibrous yarn tubing, (b) flocked hollow plastic cylindrical tubing, (c) shaped sections of flocked fabric, (d) wound flocked band or tape and (e) wound flocked yarn. These pre-flocked swab tip configurations are then suitably attached to the end of a thin cross-section swab stick, shaft or rod. In some configurations, the end of the swab stick/shaft/rod will be tapered. In some configurations, the tip of the swab stick is rounded or semi-pointed to facilitate their insertion into a tubular plastic tubing or hollow braided yarn type modular pre-flocked tip configurations. In other configurations, pre-flocked swab tips can be attached to the end of a swab stick and positioned to render adjustable amounts of flexibility to tipoff the swab. These modular pre-flocked tip material designs are adhesively coupled to the end of swab stick/shaft/rod to form the finished medical swab which is subsequently sanitized and antiseptically packaged for eventual use.

One embodiment of a bodily fluid absorbing fiber flocked layer medical swab includes:

a pre-flocked tip including, a pre-flocked fabric, a pre-flocked yarn band, a pre-flocked hollow plastic tube, a pre-flocked, hollow braided yarn tube or a pre-flocked yarn wound base. The swab device further includes a swab rod adhesively coupled to the body fluid absorbent modular pre-flocked tip This swab rod can be adhesively coupled to the flocked tip by inserting the end of an adhesive coated tapered swab stick into the pre-flocked yarn on a hollow plastic tube or hollow flocked fibrous braid, or winding the filter tip around the swab rod or taping the filter tip to the swab rod; and the swab rod includes a tapered end coupled to the flocked yarn tip or a constant diameter end coupled to the flocked tip. In various embodiments the base (flocked upon media) of the pre-flocked fabrics includes, but is not limited to: (a) a nonwoven fabric, (b) a woven fabric, (c) a knitted fabric or (d) a fabric gauze.

In one embodiment, a process to make a bodily fluid absorbing fiber flocked layer medical swab includes the step of: providing a modular pre-flocked tip, providing a swab rod, applying adhesive onto at least one of the swab rod and the modular pre-flocked tip, assembling the swab by inserting the modular pre-flocked tip onto the swab rod, fitting the modular pre-flocked tip onto the swab rod, or folding the modular pre-flocked tip onto the swab rod; and packaging and sterilizing the assembled swab.

Another bodily fluid absorbing fiber pre-flocked layer swab embodiment includes a modular pre-flocked tip and a swab rod adhesively coupled to the modular pre-flocked tip. The modular pre-flocked tip is a pre-flocked fabric, pre-flocked braided yarn hollow tube, a pre-flocked yarn band of fabric, a pre-flocked fabric band, a pre-flocked yarn tip, a pre-flocked hollow plastic tube or a pre-flocked yarn wound base. The swab rod is adhesively coupled to the modular pre-flocked tip by swab rod insertion into the hollow plastic tube of the modular pre-flocked tip, swab rod insertion into the braided yarn hollow tube of the modular pre-flocked tip, winding the pre-flocked yarn band of fabric onto an end of the swab rod, winding the pre-flocked fabric strip, ribbon or band onto an end of the swab rod or folding the pre-flocked fabric band to the swab rod. The swab rod includes a tapered end adapted to be coupled to the pre-flocked hollow plastic tube, a tapered end adapted to be coupled to the pre-flocked hollow braided yarn tube or a constant diameter end adapted to be coupled to the pre-flocked yarn tip.

In another embodiment, the modular pre-flocked tip includes an adhesive coated, die-cut, folded over, shaped, pre-flocked fabric section. The pre-flocked section includes a light weight nonwoven fabric. An unflocked side of the modular pre-flocked tip includes an adhesive coating. In this embodiment, a top portion of the modular pre-flocked tip is bendable and

flexibility of a bendable modular pre-flocked tip is adjustable by an insertion depth of the swab rod in relation to a top edge of the modular pre-flocked tip.

In one embodiment, a method of making a bodily fluid absorbing fiber pre-flocked layer medical swab includes: providing a modular pre-flocked tip and a swab rod, applying adhesive onto at least one of the swab rod and the modular pre-flocked tip, assembling the swab by one of: inserting the modular pre-flocked tip onto the swab rod; fitting the modular pre-flocked tip onto the swab rod; and folding the modular pre-flocked tip onto the swab rod. The method further comprises packaging and sterilizing the assembled swab.

In other embodiments, providing a modular pre-flocked tip includes providing pre-flocked tubing from pre-flocked plastic tubing or pre-flocked braided yarn tubing; and cutting a sectional length of the pre-flocked tubing to form a hollow tubular tip section. In yet another embodiment, the tubing is plastic tubing or braided yarn tubing and the braided yarn tubing includes nylon filament yarn or polyester filament yarn; and the yarn has a denier of about 70 to about 200.

In other embodiments, providing a swab rod includes providing a swab rod having a tapered end, and applying adhesive to the tapered end of the swab rod and assembling the swab by inserting the tapered end of the swab rod into the hollow pre-flocked tubular tip section. In other embodiments, providing a modular pre-flocked tip includes providing pre-flocked yarn, providing a swab rod comprises providing a swab rod having a tapered end and the method further includes coating the tapered end of the swab rod with adhesive and winding a section of the pre-flocked yarn around the adhesively coated tapered end of the swab rod.

In another embodiment, providing a modular pre-flocked tip includes providing pre-flocked yarn or providing pre-flocked fiber fabric having a flocked side and a non-flocked side; slitting the pre-flocked fabric into narrow bands, coating a non-flocked side of the narrow bands with adhesive. The method further includes cutting a section of the adhesively coated pre-flocked fabric narrow bands and wrapping the section of adhesively coated pre-flocked narrow band around an end of the swab rod.

In another embodiments providing a modular pre-flocked tip includes providing pre-flocked fabric, die-cutting the pre-flocked fabric into shaped sections, applying adhesive to a non-flocked side of the die-cut pre-flocked fabric shaped sections. The method further includes

folding over and adhering one of adhesively coated die-cut pre-flocked fabric shaped sections to an end of the swab rod. The method further includes folding the adhesively coated die-cut pre-flocked fabric shaped section over a top of the swab rod or folding the adhesively coated die-cut pre-flocked fabric shaped section across a side of the swab rod. In a further embodiment, the adhesive is a hot-melt adhesive, a heat sensitive adhesive or a Pressure Sensitive Adhesive (PSA) laminated directly to the non-flocked side of the die-cut pre-flocked shaped sections covered with a suitable release film. In another embodiment, the method includes adjusting a position of modular pre-flocked tip with respect to an end of the swab rod to adjust flexibility of the modular pre-flocked tip.

Such swab configurations and methods of manufacture simplify design and modular manufacturing compared to traditional flocked medical swab manufacturing. The use of “modular pre-flocked tips” fabric or flocked yarn or tape or ribbon layers to fabricate flocked swab tip configurations facilitates on-site manufacturing, since these techniques eliminate the on-site electrostatic flocking technology requirement for flocked medical swab manufacturing in-house. With these modular components, the is no need for a flocking technology specialist, an investment in a flocking facility. In-house manufacturing by technicians is accomplished in an on-demand basis for hospitals and for testing labs, and no assembled swab inventory is required.

Having these modular pre-flocked tip material fabrication and assembly techniques available for hospitals and testing labs will eliminate supply chain problems as has been encountered in the early stage of the COVID-19 outbreak. The use of certain flock fibers and yarns (e.g., hollow fibers, sea/islands bicomponent fibers, bio-material fibers, cellulose acetate, etc.) enhances biological sample collection of body fluid absorption efficiency by very significant increases in collection surface area and volume of the flocked medical swab. On-site swab fabrication technology is an improvement in medical swab availability for hospitals and testing labs to overcome severe supply chain problems.

### BRIEF DESCRIPTION OF THE DRAWINGS

The foregoing and other objects, features and advantages of the invention will be apparent from the following more particular description of embodiments of the invention, as illustrated in the accompanying drawings and figures in which like reference characters refer to the same parts throughout the different views. The drawings are not necessarily to scale, with emphasis instead being placed upon illustrating the embodiments, principles and concepts of the invention. These and other features of the invention will be understood from the description and claims herein, taken together with the drawings of illustrative embodiments, wherein:

FIG. 1A is a side view of a section of a pre-flocked hollow plastic tube swab tip before assembly in accordance with example embodiments disclosed herein;

FIG. 1B is an end view of a section of a pre-flocked hollow plastic tube swab tip before assembly of the section of a pre-flocked hollow plastic tube swab tip of FIG. 1;

FIG. 1C is a diagram of an assembled Flocked tip modular swab rod including a in accordance with example embodiments disclosed herein;

FIG. 2A is a diagram of a tapered end swab rod and a pre-flocked swab tip before final assembly in accordance with example embodiments disclosed herein;

FIG. 2B is a diagram of a bodily fluid absorbing fiber flocked layer swab in accordance with example embodiments disclosed herein;

FIG. 3 is a schematic diagram of a Yarn Flocking Device for modular flocked medical swab in accordance with one example embodiment disclosed herein;

FIG. 4 is a side view of flocked yarn for tip winding in accordance with one example embodiment disclosed herein;

FIG. 5 is a cross section end view the flocked yarn of FIG. 4;

FIG. 6 is a diagram of Double-side PSA tape/yarn band laminator in accordance with one example embodiment disclosed herein;

FIG. 7A is an assembly diagram of a flexible tip Flocked Layer Medical Swab showing a pre-flocked Fiber Layer die-cut shape and Swab rod prior to assembly in accordance with one example embodiment disclosed herein;

FIG. 7B is an assembly diagram of a top folded flexible tip Flocked Layer Medical Swab showing the swab rod placed onto adhesive treated back of Die-Cut Shape of FIG. 7A;

FIG. 7C is an assembly diagram side view of a top folded flexible tip Flocked Layer Medical Swab showing Flocked Layer folded over itself/adhesive consolidated of FIG. 7B; and

FIG. 7D is an assembly diagram side view of a top folded flexible tip Flocked Layer Medical Swab showing Flocked Layer folded over itself/adhesive of FIG. 7B.

FIGS. 8A-8D are assembly diagram of a top folded flexible tip Flocked Layer Medical Swab similar to FIGS. 7A-7D but showing a side fold design;

### DETAILED DESCRIPTION

In embodiments described below simplified modular flocked fiber swab designs are provided. The embodiments described below include four general configurations (1) flocked hollow continuous tubing; (2) flocked continuous filament yarn; (3) flocked narrow ribbon/tape shaped and (4) “pre-flocked” (foldable) planar, fabric layer die-cut section configurations which are used as the starting tip material for collecting medical bio-fluids test specimens. These pre-flocked fiber materials form the “head”, tip or “bulb” of the swab. These pre-flocked swab tips are assembled on a suitable swab rod of length and diameter required for either nasopharyngeal (NP) or oropharyngeal (OP) specimen collection device for test kit inclusion/application. In other applications the length of the rod and size of the modular pre-flocked tip varies according to needs and applications.

Generally defined flock materials and adhesives for flocked medical swabs provided herein are:
(1) Flocked Fiber Materials: The types and nature of the flock fiber material that can be used for the invented designed swabs are very wide, versatile and various. Standard, nylon and polyester flock fibers of various denier, and length are all certainly available of use. Many other types of fiber, primarily synthetic fiber, are also applicable, namely,
(a) hollow fibers,
(b) tri-lobal fiber,
(c) sea/islands bi-component fibers, post-split-able (fibrillated) fibers,
(d) Cellulose acetate fibers and the like; and
(e) Keratin, Alginate or other bio-polymer fiber materials.

(2) Adhesives for Swab Fabrication: There are a number of adhesive types and forms that are used in the “on-site” preparation of these invented swabs. Pressure Sensitive Adhesives (PSA) like adhesives are convenient by designing the adhesive to have a strong bonding aggressiveness of the PSA of sufficient strength. Another type of adhesive is a radiation curable adhesive (e.g., an Ultra-Violet (UV) or LED radiation curing adhesive). However, UV and LED curing adhesives are subject to cure-through problems. In this regard, a UV curing/Heat curing epoxy resin adhesive (UV15DC80MED) is a commercially available adhesive from Master-Bond Products, Hackensack, N.J. This Master-Bond adhesive as well as other available radiation curable adhesives are readily adaptable to bonding the flocked material to the tip of the flock stick or shaft.
(3) In-House swab fabrication and production capabilities: in several embodiments the flocked swab tips can be prefabricated and finished flocked medical swabs are assembled in a machine designed, robotic-like component assembler (e.g., Pick- and Place assembler) for final production of the flocked medical swabs. Alternatively, the swabs are assembled manually. In one machine processed embodiment, the production capacity of this designed swab assembly system is in the range of 6000 to 12000 swabs per hour, while for manual operation the range is 2000 to 5000 swabs or more per day. With the swab design and assembly process, provided herein, the end user/in-house operator does not need electrostatic flocking and other complicated processing equipment or techniques to fabricate or manufacture the medical swabs for test kits. The modular pre-flocked tip component material is pre-fabricated by a commercial flocked material fabricator and custom supplier. Using these pre-flocked components, the healthcare facility and the staff members in testing labs and hospitals are able to fabricate medical swab devices for in-house use as needed employing an assembly methodology that resembles a 3D printer-like pick and place system. These swab articles are then sanitized, antiseptically packaged and rendered useable in COVID-19 or other bacterial or viral evaluating medical test kits.

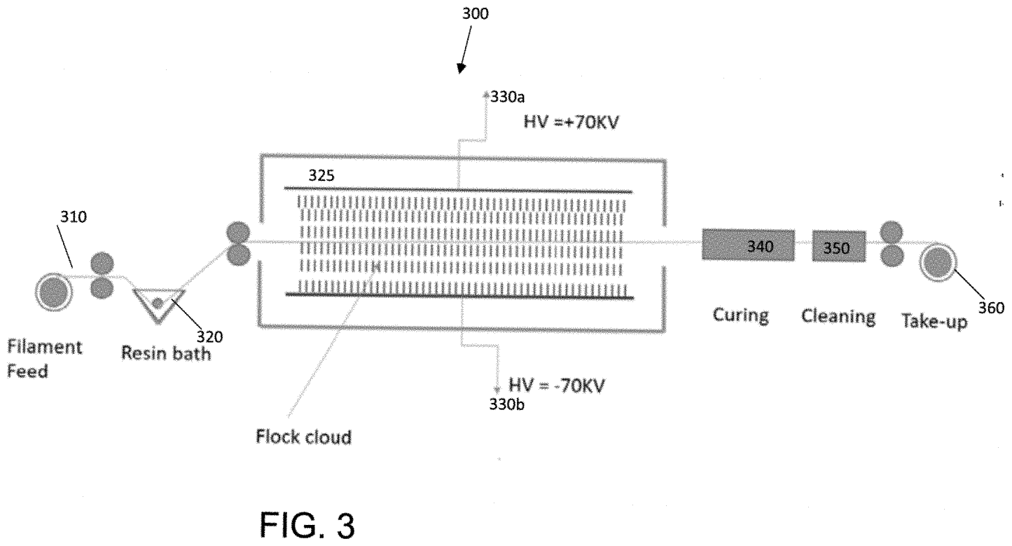

It is understood, that the steps to fabricate the swab can include the flocking process or alternatively the swab assembly can begin with the use of modular pre-flocked tips manufactured from fabric, yarns, tubing and other pre-flocked materials. Some of the embodiments below include details of the flocking process using loose fibers and a flocking machine shown below in conjunction with FIG. 3.

### Flocked Plastic Tube or Flocked Braided Yarn Flock Tips Designs

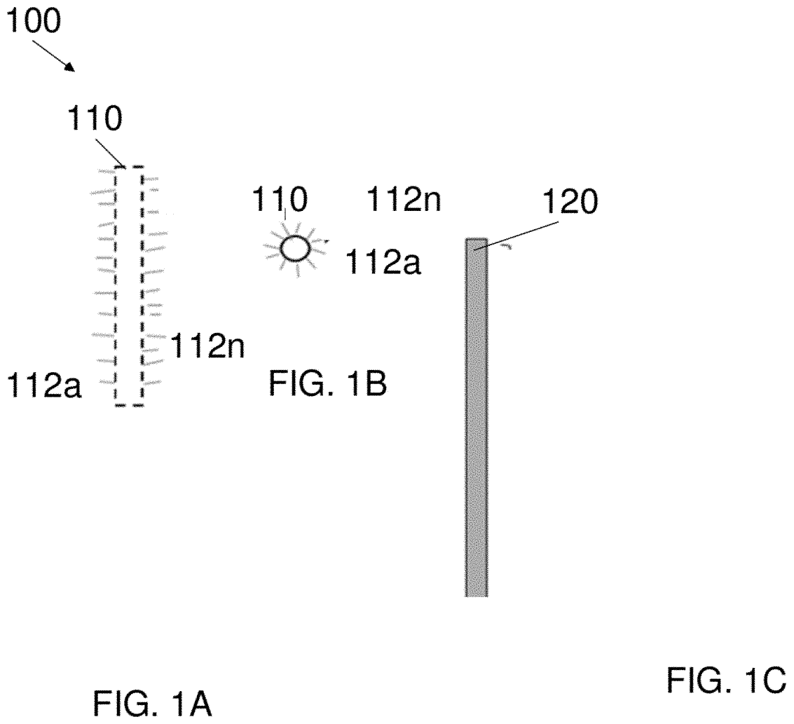

Now referring to FIG. 1A showing a side view, a pre-flocked hollow plastic tube (or optionally a braided tube) modular pre-flocked tip 100 includes a section 110 of a tube, here a plastic tube, the outer surface of which has been flocked with body-fluid absorbing flock fibers 112a-112n. In one embodiment, the modular pre-flocked tip 100 is composed of a thin, hollow, semi-flexible hollow plastic tube. FIG. 1B is an end view of the modular pre-flocked tip 100. FIG. 1C is a side view of a swab rod 120. In production, a continuous length of a thin walled, fine (e.g., 1.5 mm OD, 1 mm diameter hollow) plastic tubing is obtained from a commercial extruded plastic material supplier. Next, this thin plastic tubing is continuously flocked upon to form a “pre-flocked” hollow tubing material component. FIG. 3 below is a diagram showing this continuous “flocking onto tubing” process. This flocked plastic tubing is then cut into specific lengths to form the modular pre-flocked tips. The final swab assembly is accomplished by inserting and securing, using an adhesive, the end of the swab rod into the hollow cavity of the modular pre-flocked tip. In some embodiments the swab rod has a tapered end to facilitate insertion into the hollow tube of the modular pre-flocked tip. In certain embodiments the tube, also referred to a filament, can be yarn; a hollow plastic tube, braided flexible tube or hollow braided yarn. In production, the modular pre-flocked tip can be made from flocked continuous hollow tubing. In other embodiments the tube base can be supplied as either a plastic tube, braided flexible tube or as a braided yarn tube. In one embodiment the tube is a hollow braided flexible tube produced on an 8-16 carriage Braiding Machine, with nylon or polyester of 70 denier to 200 denier filament yarns. A typical OD is about 0.8-1.0 mm and a typical ID is about 0.3-0.7 mm. In one embodiment, the hollow braided flexible tube includes nylon polyester flock fibers having a size of 0.5-2.0 mm OD, a density of 70 denier to 200 denier.

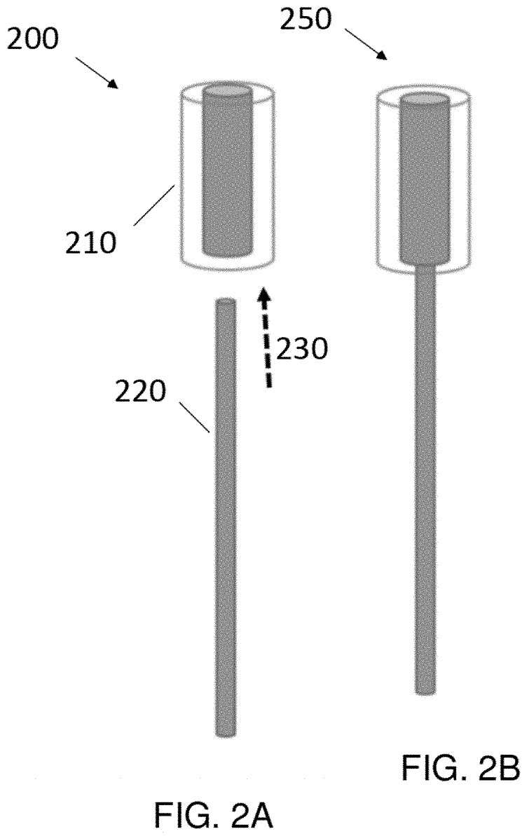

Now referring to FIG. 2A, an unassembled flocked 200 includes a modular pre-flocked tip 210 and a swab rod 220 (also referred to as a swab handle, stick of shaft 220). An optional tapered swab rod is not shown. FIG. 2B shows an assembled bodily fluid absorbing fiber flocked layer swab 250. Arrow 230 shows the insertion direction of the swab rod 220 into the modular pre-flocked tip 210.

Now referring to FIG. 3 a flocking device for modular flocked swabs 300 includes tubing feeder 330, a resin bath 320, yarn flocking box 325, a high voltage supply across electrodes 330a and 330b, a curing module 340, a cleaning module 350 and a take up spool 360. FIG. 3 is an illustration of a method of fabricating pre-flocked thin plastic tubing or hollow braided tube which is a precursor for making a modular pre-flocked tip.

A first embodiment, here the Flocked Plastic Tube, uses the following the steps for making a tubular modular pre-flocked tip for a NP or an OP swab:
Step A1) Provide thin cross-section hollow plastic tubing of OD equals 0.5 mm-1.5 mm and ID equals 0.3 mm-1.25 mm and at least 15 m long on a spool as substrate material flocked “yarn;” cylindrical tubing.

Step A2) A braided yarn or thin cross-section plastic tubing flocking machine 300 (FIG. 3) is used to flock the tubing. The flocking machine 300 includes a flexible hollow tubing feeding spool 310, resin bath 320, yarn flocking box 325, UV, LED or heat curing station 340, vacuum cleaning 350 and take-up spool 360.
Step A3) Provide Flock fibers: Polyester (PET, Dacron®), nylon, cellulose acetate, viscose or PET sea-island bicomponent fibers; fiber fineness is generally in the range of 0.5 to 3 denier; flock fiber length is generally in the range of 0.3 mm to 5 mm Typically, 0.5 mm flock lengths are used on 1.5 mm OD tubing.
Step A4) Set up the textile yarn or thin hollow plastic tubing flocking device with selected flock fiber in the step A3 above, and flock adhesive (UV curable, water base etc.) in resin bath. Application thickness is controlled by a nozzle die (diameter=tubing OD+0.1 mm, typical for 0.5 mm flock length);
Step A5) Flock density (100-500 fibers per mm²) is controlled by flocking High Voltage on two electrodes 330a and 330b for generating a flock “cloud” in the flocking chamber. The voltage is typically ±50 to 80 KV at a 20 cm electrode separation, and take up speed (i.e., residence time in the flocking zone) is approximately 20 cm per minute for this embodiment and is a function of a flocking zone length, here 50 cm.
Swab Final Assembly for The Cylindrically Formed Modular pre-flocked tip
Step A6) Depending on the application, select polymeric (e.g., for medical applications) or wooden swab rod, typical 2-2.5 mm diameter×150 mm, tapered 1.5 mm×70 mm long, apply epoxy or suitable adhesive of about 10 mm on the tapered end.
Step A7) Cut the 15 mm long tubular tip from the spool of flocked hollow tubing prepared in steps A1-A5. Typical tip geometry is outer diameter 2.5 mm×overall tip length 15 mm
Step A8) Fit the tubular tip snuggly on to the adhesive applied tapered end of the rod, and cure with heat or UV or LED radiation. Alternatively, this assembly operation is achieved on a pick and place robotic assembly machine with a rod holding tray.
Step A9) Package and sterilize the assembled swabs. See step E5 below for further details on sterilization.

### Flocked Continuous Filament Yarn

In a second embodiment a wound modular pre-flocked tip is made from flocked fibers deposited onto continuous filament yarns.

A flocked filament yarn wound tip base material for NP or OP swab is made using the following process:
Step B1) Provide polyester or nylon multi-filament yarn of 100-150 tex (where 1 tex equals a linear density of 1 gram per 1000 m) with regular twist on a spool as substrate material flocked “yarn”. For example, yarn diameter of 150 tex regular twist PET multifilament equals a 0.48 mm according to the formula: d (cm) equals T 0.5/(280.2) (φρf) 0.5, where T (yarn size in tex), φ (packing factor of the yarn), ρf (fiber density).
Step B2) Yarn flocking is achieved on a yarn flocking machine (FIG. 3) which includes a filament feeding spool, resin bath, yarn flocking box, UV, LED or heat curing station, cleaning and take-up/winding roll. Alternatively flocked yarn of desired diameter with specific is commercially available flock fibers.
Step B3) Provide Flock fibers: Polyester (PET, Dacron®), nylon, cellulose acetate, viscose or PET sea-islands bicomponent fibers; fiber fineness=0.5 to 3 denier; fiber length=0.3 mm to 5 mm Typical 0.3 mm to 0.5 mm flock length are used for multifilament flocked yarn tip materials.
Step B4) Set up the yarn flocking device with selected flock fiber in the section 3 above, and flock adhesive (UV or LED curable, water base etc.) in resin bath. Application thickness is controlled by a nozzle die (diameter=filament yarn diameter+0.1 mm, typical for 0.5 mm flock length)
Step B5) Control Flock density (100-500 fibers/mm²) by flocking High Voltage on two electrodes for generating flock “cloud” in the flocking zone (typically dual polarity of ±50 to 80 KV at 20 cm electrode separation) and take up speed (i.e., residential time in the flocking zone).

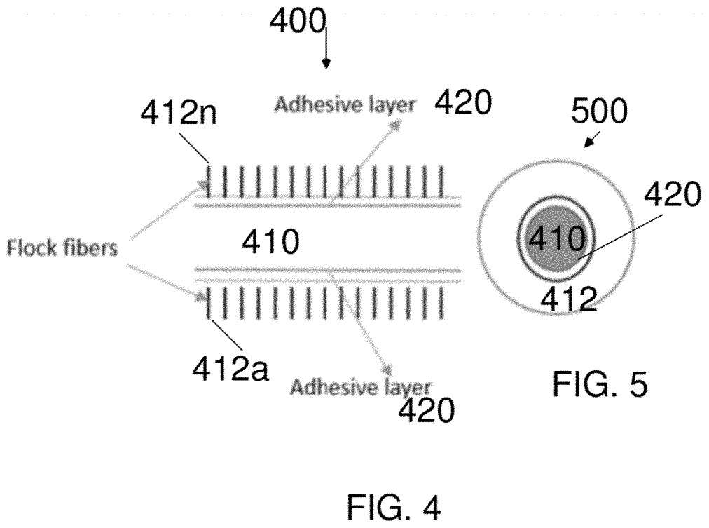

Now referring to FIG. 4, the flocked filament yarn for tip winding 400 includes a textile yarn core 410, an adhesive layer 420 disposed adjacent to the textile yarn core 410, and a plurality of flock fibers 412a-412n (collectively referred to as flock fibers 412) which are flocked onto the adhesive layer 420.

### Wound Flocked Yarn Medical Swab Final Assembly

Step B6) Select a plastic or wooden swab rod, typically 1.5 mm diameter OD×150 mm length to about 2.5 mm OD×80 mm length handle with tapered end of about 1.5 mm OD×70 mm length; apply epoxy or suitable adhesive on the tapered end to about a 10 mm length along the swab stick end.
Step B7) Prepare the flocked fibrous yarn (1.1 mm outer diameter) with 0.3 mm flock on 150 tex PET multifilament yarn is on a spool.
Step B8) Make a flocked yarn tip, winding the yarn from the spool closely 15 turns with appropriate tension on the adhesive applied tapered end of the swab rod, and finally curing with heat or UV. This provides a flocked yarn swab tip about 3 mm OD×15 mm in length.
Step B9) Packaging and sterilizing of the assemble swabs. See step E5 below for further details on sterilization.

### Flocked Narrow Ribbon/Tape Shaped Modular Pre-Flocked Tips

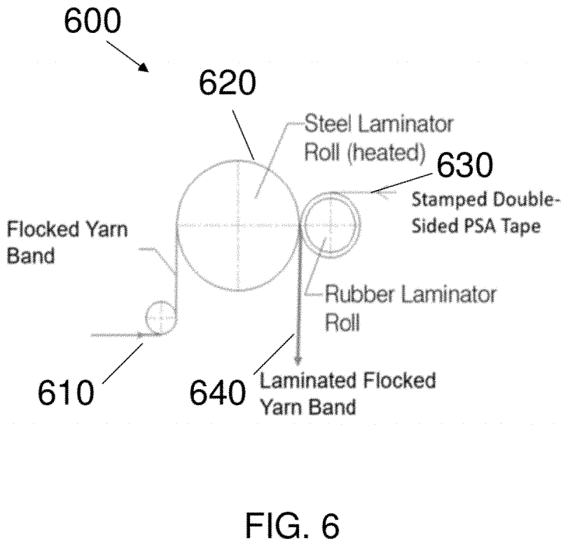

In a third embodiment, a pre-flocked fabric band swab tip design is used. In this embodiment, lengths of pre-flocked fiber fabric are obtained from a flocked material vendor and used for the body-fluid absorbing tip of the medical swab. Typical flocked fabric configurations, include for example, 1.0 mm long flock on 150 tex PET multi filament yarns making a cylindrical tip from the flocked yarn band.
Step C1) Provide pre-flocked filament yarns obtained from commercial supplier has OD=1.1 mm with 0.3 mm flock on 150 tex PET multifilament yarn on a spool.
Step C2) Winding five spools of the pre-flocked filament yarns on a spool of 4.4 mm (W)×250 mm (Flange) under tension tightly to form a flocked yard band.
Step C3) Coat on side of the yarn band with suitable grade adhesive, and cure by heat or UV to make yarn bands on a spool.
Step C4) Prepare double PSA sticky tape (6.4 mm wide) of band aid grade with one side has indentation mark for peeling at the 4.4 mm from the left edge.
Step C5) Peel off a layer of 4.4 mm from one side of the double-sided PSA tape and laminate with 4.4 mm wide flocked yarn band on the laminating machine (FIG. 6.)

Now referring to FIG. 6. A double-side PSA tape/yarn band laminator 600 includes feed roller 610, heated steel laminator roller 620, rubber laminator roller 630. Roller 630 feeds stamped double sided PSA tape which is laminated with the flock yarn band to produce laminated flocked yarn band.
Step C6) The double-side PSA tape (¼″ or 6.4 mm)/flocked yarn band (tape) laminates are ready for flocked yarn tip fabrication to make the flocked yarn band medical swab assembly;
Step C7) Cut the flocked yarn band to a length of 15 mm;
Step C8) Peel-off and wrap 6.4 mm of the band on a swab rod (also referred to as a swab handle) having a 1.5 mm OD. Making sure that the 2.4 mm overlap is free of gaps of flocked surface on the tip. The swab rod can have any of 1) a tapered end, 2) a reduced diameter from the handle or 3) a constant diameter. This completes the 15 mm long about 2.5-3 mm OD cylindrical NP Flocked medical swab.
Step C9) Packaging and sterilizing of the assemble swabs. See step E5 below for further details on sterilization.

### Pre-Flocked Foldable Planar, Fabric Layer Die-Cut Section

The pre-flocked foldable planar, fabric layer die-cut section design includes three embodiments described below starting with a fourth embodiment.

In a fourth embodiment, a flocked medical swab is made from flocked fabric. In this embodiment, pre-flocked fabrics are obtained from a flocked material vendor and are used to assemble the bodily-fluid absorbing tip of the medical swab. Typical flocked fabrics purchased are 0.5 to 2 m, 1 to 10 denier long PET or nylon flock on PET nonwoven substrate fabrics of 20 to 100 g per m². A suitable nonwoven fabric is Pellon #810 Tru-Grid type, 100% PET nonwoven fabric.

Fabrication of Modular Pre-Flocked Tip Assemblies from Pre-Flocked Fabric
Step D1) Obtain flocked nonwoven fabric from a commercial manufacturer having the following specifications 1 mm, 3 denier nylon flock onto the thin PET nonwoven fabric base. Have the manufacturer supply the fabric in suitable widths (such as 50 to 60 inches) whereby the flocked nonwoven fabric is back-coated with a suitable medical grade pressure sensitive adhesive (aggressive quality PSA adhesive). The flocked nonwoven fabric will thus be supplied having a release-film back-up coating layer.
Step D2) Prepare double-sided PSA tape (¼″ or 6.4 mm wide) of band aid grade PSA adhesive with one side has indentation mark for peeling at the 4 mm from the left edge.
Step D3) Peel off 4.4 mm from one side of the double-sided PSA tape and laminate on the non-flocked back side of the flocked “tape” with 4.4 mm wide flocked fabric band on the laminating machine (FIG. 6.)
Step D4) The double-side PSA tape (¼″ or 6.4 mm)/flocked yarn band laminates are ready for flocked yarn tip fabrication.

### Flocked Fabric Band Medical Swab Assembly

Step D5) Cut the flocked fabric laminated band 15 mm long Step D6) Peel-off 2.4 mm and 6.4 mm side and wrap 6.4 mm on a swab handle of 1.5 mm OD. Assure that the 2.4 mm overlaps to have no gaps of flocked surface on the tip.
Step D7) Packaging and sterilizing of the assemble swabs completes Flocked medical swab of about 15 mm long and about 2.5-3 mm OD cylindrical NP. See step E5 below for further details on sterilization. It is understood that the swab tip can have various shapes. Here the tip has a bulbous shape.

In a fifth embodiment, a swab is made from pre-flocked fabric Symmetrically Folded upon itself and locked onto the end of a swab stick/shaft/rod/handle:
Step E1) Provide pre-flocked flock fabric (e.g., bulk custom pre-flocked fabric from custom flocking companies such as Spectro Coatings, Leominster, Mass. or Cellusuede Products Company, Rockford, Ill. As appropriate these custom flock fabric converters could also coat the back of these Custom Flocked fabrics with a Hot-Melt elastomeric of other heat sensitive adhesive or the like. Another configuration employs Pressure Sensitive Adhesive (PSA) laminated directly to the back side of the flocked fabric and covering this PSA coated backside with a suitable release film. Another variation one side of available double-sided PSA tape adhered to the backside of the flocked fabric. Pressure-sensitive adhesive back flocked fabrics are used in the retail market sewing craft field. Medical grade PSAs are readily available such as those used in “band-aid” type finger bandages.
Step E2) The custom pre-flocked fabric is die-cut into numerous individual, pre-flocked swab tips shaped sections.
Step E3) In this embodiment a swab is created by side-folding a small die-cut shaped section of Pressure Sensitive Adhesive (PSA) back flocked fabric material. A key difference in this design is that the side folded flocked layer shape is attached to the end of the swab-stick such that the flocked fabric material deliberately over-laps the end of the swab stick. When this flocked layer fabric shape is folded over the swab stick/shaft/rod/handle end and pressed together to secure the swab tip to the swab stick end, this overlapped small section of the flocked fabric die-cut flocked fabric shape is not supported by the stiff material swab stick/shaft/rod/handle, to create an unsupported zone. The swab material end is then rendered bendable or flexible during the functioning of the medical swab itself. The flocked swab material fabric is not supported by the swab stick materials in this unsupported zone. This is a unique feature of this particular embodiment; when the swab system is used to collect the medical specimen from the tested patient, the bendable/flexible end of the bendable tip swab has a softer contact with the human tissues that contact the medical bio-fluid the swab; provides more comfort to the patient being tested patient. Furthermore, this bendable-end swab tip design provides a greater area the swab contacting the infected human tissue area. The gives this swab design added effectiveness in collecting the bio-fluid test sample.

This area or overlapped zone of the flocked fabric is then rendered uniquely flexible, and easily bends under the swabbing pressure to substantially conform to the complex shape of inside the nose, mouth, throat, rectum (i.e., the body cavity part for obtaining the swab sample).

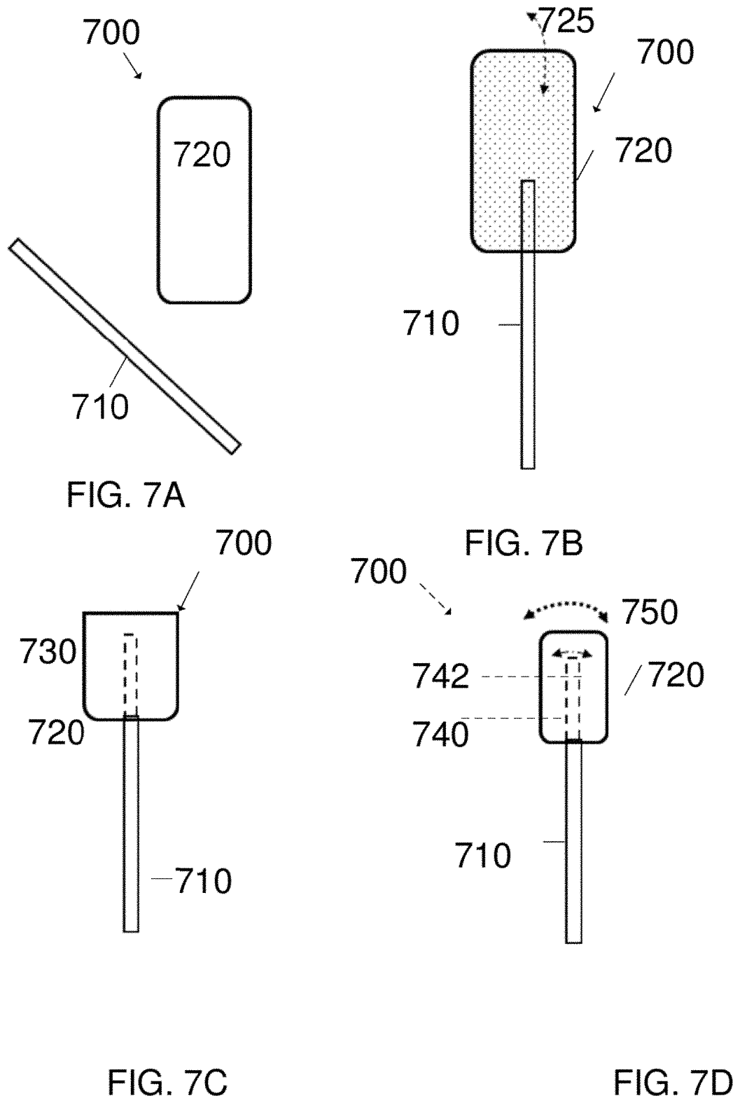

Now referring to FIG. 7A, a bendable/flexible tipped flocked swab, 700 includes a swab rod 710, and a pre-flocked fabric shaped section 720. Arrow 725 shows the pre-flocked fabric shaped section 720 folding over the top of the swab rod 710 during manufacture. The pre-flocked fabric shaped section 720 is adhesively attached to the swab rod in FIG. 7B and the pre-flocked fabric shaped section 720 is adhesively coated and is shown folded over the swab rod 710 in FIG. 7C. Different ranges in pre-flocked swab tip end flexibility (as indicated by arrow 750) shown in FIG. 7D are achieved by different placement of the end of swab rod 740 with respect to the pre-flocked fabric shaped section 720, for example, at positions 740 or 742. It is noted that the swab rod does not extend to the end or top of modular pre-flocked tip (i.e., the layer of flocked fabric that constitutes the final flocked fabric swab tip).

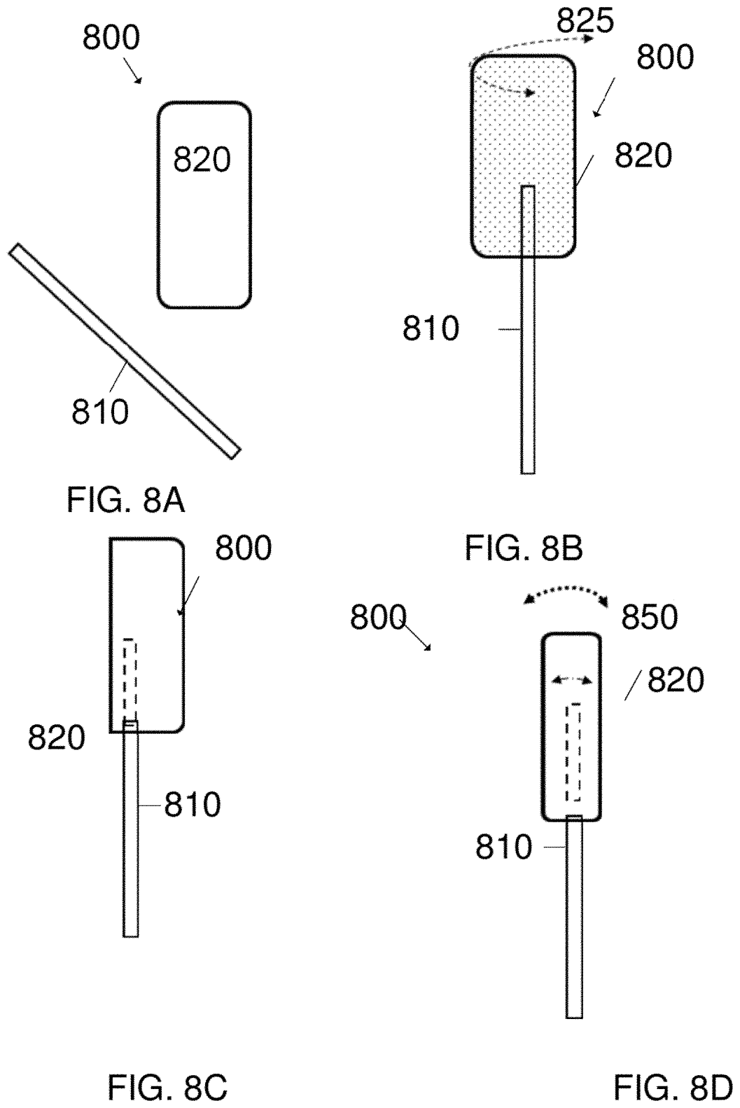

FIGS. 8A-8D show a side folded bendable/flexible tipped flocked swab 800 to the bendable/flexible tipped flocked swab 700 of FIG. 7. Arrow 825 shows the pre-flocked fabric shaped section 820 folding across the side of the swab rod 710 during manufacture.
Step E4) Alternatively, if a hot melt adhesive is used as the back-of-the-flocked-fabric bonding material, this fold-over the tip of the swab stick/shaft/rod/handle step E4 is secured by applying heat and pressure to the folded die-cut swab tip preform. In other words, this flocked fabric tipped swab end now folded to about ½″×1″ long (alternatively about 4 mm×15 mm) is placed into a heated anvil, fixed shape pressing jig so that the shaped, folded flock layer strip is pressed together and consolidated and adhered to the end of the swab stick. In other words, the flocked fabric die-cut shape is consolidated and the folded fabric layer is bound together onto the tip of the swab stick by the available hot melt adhesive system.

The final configuration then takes the form of a small shaped rounded edge rectangle of flocked fabric (like a “fuzzy” spatula) at the end of a thin, semi-stiff stick or shaft.
Step E5) This flocked surface swab device is then submitted into a disinfecting chamber (ethylene oxide, heat, hydrogen peroxide) to render it disinfected and the antiseptically packaged for later medial sample collection use. In one embodiment the folded flock fabric layer designed swabs are rectangular in shape having semi-oval or rounded corners, and are used as a mouth swab or tongue swab. This design would be like a “spatula” shaped swab. Other designs of this flocked fabric swab tip are also feasible. Some of these configurations are:
(a) Flocked Fabric “Wraparound” design in which a section of the flocked fabric is rolled onto the end of the swab shaft/rod/stick,
(b) a thin ribbon of flocked fabric is rolled/wrapped (i.e., a “barber-pole” wrap) around the end of a swab-shaft/stick/rod.

In a sixth embodiment similar to the fifth embodiment, a swab is made from “pre-flocked” fabric Side-Folded, Bendable/Flexible-Tip Swab Design. This embodiment is similar to the fifth embodiment above with step F3 replacing step E3.
Step F3) Starting with pre-flocked fabric, pre-flocked yarn, pre-flock band or pre-flocked, die-cut, shaped swab sections assembled into tips and swab rods (tapered or not tapered) there are several ways to assemble the final swab described below.

Automated (Robotic) Swab Assembly: Specialized robotic, component manipulating assembly machines of various complexity are used to automatically assemble the fabricated fiber flocked swab tip and rod assembly.

Manual Swab Assembly: Here, one starts with a pre-manufactured supply of flocked swab tips and swab sticks/rods. It is possible then to assemble these swab components by hand at a hospital or medical care facility (on-site). For example, certain swab embodiments could be assembled by hand by hospital personnel such as a nurse or hospital laboratory technician as needed before use. This assembly of a swab-by-hand methodology would be most adaptable to the die-cut, flocked fabric. A swab assembly person just has to carefully apply the die-cut flocked fabric pre-designed section piece to the end of the swab stick/rod by first peeling off the release paper from the back of the flocked fabric swab tip section to expose the PSA. This die-cut piece is then carefully folded over onto the end of the swab stick/rod to form the flocked fabric swab tip. Finger pressure would then be applied to secure or lock the PSA treated flocked fabric swab tip section to the end of the swab stick/rod. Bendable or non-bendable tip swabs can hereby be fabricated depending upon the position (distance from the end of the swab stick/rod) the flocked fabric section is secured to the swab stick/rod. These assembled flocked swabs are then sanitized and antiseptically packaged (as needed) for future medical sample collection use. The swab components could also be pre-sanitized.

In certain embodiments, a yarn swab tip made from flocked fibers deposited onto continuous light weight planar nonwoven, woven or gauze fabric. The modular pre-flocked tip made using flocked fabric has the following features: The modular pre-flocked tip or swab tip can have various shapes, sizes, thicknesses and geometries. These variations depend upon the length of flock used and the shape and size of the flocked swab tip end-tab. There are nasal swabs, oral swabs, anal swabs and ointment coating swabs and non-medical cosmetic swabs; each has design/shape/size requirements.

The type and treatment of the flock fiber used in fabricating the flocked fabric medical swabs is also an important varying parameter. The employment of bi-component nano-fibrillate-able flock fibers could create very high fibrous tipped swabs. Additionally, crimped cellulose acetate fibers can be flocked onto a base fabric. One important feature of these flocked fabric swab tip designs is the ability to now create flexible tipped swab designs. This feature is easily created by off-set placing the modular pre-flocked tip material element away from the tip of the swab rod or shaft.

The section headings used herein are for organizational purposes only and are not to be construed as limiting the subject matter described in any way. While the present invention has been described in conjunction with various embodiments and examples, it is not intended that the present teachings be limited to such embodiments or examples. On the contrary, the present invention encompasses various alternatives, modifications, and equivalents, as will be appreciated by those of skill in the art. While the teachings have been particularly shown and described with reference to specific illustrative embodiments, it should be understood that various changes in form and detail may be made without departing from the spirit and scope of the teachings. Therefore, all embodiments that come within the scope and spirit of the teachings, and equivalents thereto are claimed. The descriptions and diagrams of the methods of the present teachings should not be read as limited to the described order of elements unless stated to that effect.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

We claim:

1. A bodily fluid absorbing fiber pre-flocked layer swab comprising:
a modular pre-flocked tip; and
a swab rod adhesively coupled to the modular pre-flocked tip.

2. The bodily fluid absorbing fiber pre-flocked layer swab of claim 1, wherein the modular pre-flocked tip comprises at least one of:
a pre-flocked fabric;
a pre-flocked braided yarn hollow tube;
a pre-flocked yarn band of fabric;
a pre-flocked fabric band;
a pre-flocked yarn tip;
a pre-flocked hollow plastic tube; and
a pre-flocked yarn wound base.

3. The bodily fluid absorbing fiber pre-flocked layer swab of claim 2, wherein the swab rod is adhesively coupled to the modular pre-flocked tip by one of:
swab rod insertion into the hollow plastic tube of the modular pre-flocked tip;
swab rod insertion into the braided yarn hollow tube of the modular pre-flocked tip;
winding the pre-flocked yarn band of fabric onto an end of the swab rod;
winding the pre-flocked fabric strip, ribbon or band onto an end of the swab rod, and
folding the pre-flocked fabric band to the swab rod.

4. The bodily fluid absorbing fiber pre-flocked layer swab of claim 2, wherein the swab rod includes one of:
a tapered end adapted to be coupled to the pre-flocked hollow plastic tube;
a tapered end adapted to be coupled to the pre-flocked hollow braided yarn tube; and
a constant diameter end adapted to be coupled to the pre-flocked yarn tip.

5. The bodily fluid absorbing fiber pre-flocked layer swab of claim 1, wherein the modular pre-flocked tip comprises an adhesive coated, die-cut, folded over, shaped, pre-flocked fabric section.

6. The bodily fluid absorbing fiber pre-flocked layer swab of claim 5, wherein the pre-flocked section comprises a light weight nonwoven fabric.

7. The bodily fluid absorbing fiber pre-flocked layer swab of claim 5, wherein an unflocked side of the modular pre-flocked tip comprises an adhesive coating.

8. The bodily fluid absorbing fiber pre-flocked layer swab of claim 5, wherein a top portion of the modular pre-flocked tip is bendable.

9. The bodily fluid absorbing fiber pre-flocked layer swab of claim 5, wherein flexibility of a bendable modular pre-flocked tip is adjustable by an insertion depth of the swab rod in relation to a top edge of the modular pre-flocked tip.

10. A method of making a bodily fluid absorbing fiber pre-flocked layer medical swab comprising:
providing a modular pre-flocked tip and a swab rod;
applying adhesive onto at least one of the swab rod and the modular pre-flocked tip;
assembling the swab by one of:
inserting the modular pre-flocked tip onto the swab rod;
fitting the modular pre-flocked tip onto the swab rod; and
folding the modular pre-flocked tip onto the swab rod; and
packaging and sterilizing the assembled swab.

11. The method of claim 10, wherein providing a modular pre-flocked tip comprises:
providing pre-flocked tubing from one of:
pre-flocked plastic tubing; and
pre-flocked braided yarn tubing; and
cutting a sectional length of the pre-flocked tubing to form a hollow tubular tip section.

12. The method of claim 11, wherein the tubing is plastic tubing;
wherein the braided yarn tubing comprises one of:
nylon filament yarn; and
polyester filament yarn; and
wherein the yarn has a denier of about 70 to about 200.

13. The method of claim 11, wherein providing a swab rod comprises providing a swab rod having a tapered end; and
further comprising:
applying adhesive to the tapered end of the swab rod; and
assembling the swab by inserting the tapered end of the swab rod into the hollow pre-flocked tubular tip section.

14. The method of claim 10, wherein providing a modular pre-flocked tip comprises providing pre-flocked yarn;
wherein providing a swab rod comprises providing a swab rod having a tapered end; and
the method further comprises:
coating the tapered end of the swab rod with adhesive; and
winding a section of the pre-flocked yarn around the adhesively coated tapered end of the swab rod.

15. The method of claim 10, wherein providing a modular pre-flocked tip comprises:
providing one of:
pre-flocked yarn; and
pre-flocked fiber fabric having a flocked side and a non-flocked side;
slitting the pre-flocked fabric into narrow bands;
coating a non-flocked side of the narrow bands with adhesive; and
the method further comprising:
cutting a section of the adhesively coated pre-flocked fabric narrow bands; and
wrapping the section of adhesively coated pre-flocked narrow band around an end of the swab rod.

16. The method of claim 10, wherein providing a modular pre-flocked tip comprises:
providing pre-flocked fabric;
die-cutting the pre-flocked fabric into shaped sections;
applying adhesive to a non-flocked side of the die-cut pre-flocked fabric shaped sections; and the method further comprises:
folding over and adhering one of adhesively coated die-cut pre-flocked fabric shaped sections to an end of the swab rod.

17. The method of claim 16, wherein folding over one of the adhesively coated die-cut pre-flocked fabric shaped section comprises one of:
folding the adhesively coated die-cut pre-flocked fabric shaped section over a top of the swab rod; and
folding the adhesively coated die-cut pre-flocked fabric shaped section across a side of the swab rod.

18. The method of claim 16, wherein the adhesive is one of:
a hot-melt adhesive;
a heat sensitive adhesive; and
a Pressure Sensitive Adhesive (PSA) laminated directly to the non-flocked side of the die-cut pre-flocked shaped sections covered with a suitable release film.

19. The method of claim 16 further comprising adjusting a position of modular pre-flocked tip with respect to an end of the swab rod to adjust flexibility of the modular pre-flocked tip.
